(19) United States
(12) Patent Application Publication
(10) Pub. No.: US 2001/0053891 A1
(43) Pub. Date: Dec. 20, 2001

(54) Stacked microneedle systems

(76) Inventor: Donald E. Ackley, Cardiff, CA (US)

Correspondence Address: Thomas O. Hoover, Esq., HAMILTON, BROOK, SMITH & REYNOLDS, P.C., Two Militia Drive, Lexington, MA, 02421-4799, US

(21) Appl. No.: 09/747,768

(22) Filed: Dec. 22, 2000

Related U.S. Application Data
(60) Provisional application No. 60/174,023, filed on Dec. 30, 1999.

(51) Int. Cl. A61M 5/00

(52) U.S. Cl. 604/191

(57) ABSTRACT

The present invention relates to microneedle arrays in stacked configurations for fluid delivery, including for example, chemical delivery, sensing, combinatorial chemistry, fluid injections, and microfluidic connectors.

### RELATED APPLICATION(S)

This application claims the benefit of U.S. Provisional Application No. 60/174,023, filed on Dec. 30, 1999, the entire teachings of the above application being incorporated herein by reference.

### BACKGROUND OF THE INVENTION

A common technique for chemical delivery, such as, for example, delivering drugs across or into biological tissue, is the use of needles. The needles include standard syringes or catheters. While effective for the purpose of delivering drugs, needles generally cause pain, local damage to the skin at the site of insertion, bleeding, and a wound sufficiently large to be a site of infection.

Attempts have been made to design alternative devices for active transfer of drugs, such as transdermal patches, but these devices are impractical for drug delivery that does not rely on diffusion. There still remains a need for better drug delivery devices and chemical delivery devices in general.

### SUMMARY OF THE INVENTION

The present invention includes a plurality of needles having channels aligned along common axes. The microneedle arrays in stacked configurations can be used for chemical delivery, sensing, combinatorial chemistry, fluid injection and microfluidic connectors.

In a particular embodiment, reusable interfaces are formed by connecting or stacking the microneedles. In an alternate embodiment, multiple levels are permanently laminated together to form complex injection systems. By using laminated structures in, for example, Kapton or Mylar, fluidic systems can be integrated with microneedle arrays with consistent processes.

A preferred embodiment of the present invention includes a method for fabricating tapered metal microneedles using techniques such as, for example, but not limited to, laser drilled Kapton, controlling the needle taper and dimensions, and combining the needles with laminated fluidics structures. A variety of fluidic injection functions can be performed efficiently at low cost.

By connecting or stacking the needle arrays in accordance with the present invention, complex fluidic functions, reusable connections, and long-term injections can be achieved. The present invention systems can be used for drug delivery, combinatorial synthesis, integrated sensors, battlefield monitoring, and treatment of soldiers, integrated delivery and sensor systems, controlled chemical reactions, spotting for DNA chips and proteomics and color printing.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### BRIEF DESCRIPTION OF THE DRAWINGS

FIG. 1 is a cross-sectional view of a stacked microneedle system for multiple injection of fluids.

FIG. 2 is a cross-sectional view of a stacked microneedle system having concentric needles.

FIG. 3 is a view of removably stacked dispensing microneedles.

FIG. 4A is a view of a stacked needle array system.

FIG. 4B illustrates an exploded perspective view of the stacked needle array of FIG. 4A.

FIG. 5 is a view of a stacked needle array system for multiple injections.

FIGS. 6A and 6B are views of a stacked needle array system for laminar injection systems.

FIG. 7 is a view of a replaceable sensor module.

FIG. 8 is a view of a stacked secondary array coupled to a piercing array.

FIGS. 9A and 9B are views of a microfluidic connector.

FIGS. 9C and 9D illustrate exploded views of a microfluidic system.

FIG. 9E illustrates an alternate embodiment of a fluidic connector.

FIG. 10 is a cross-sectional view of a device that combines sensing and delivery functions.

### DETAILED DESCRIPTION OF THE INVENTION

Microneedles, in particular, connected or stacked microneedles form devices which can be used, for example, to form complex injection systems. Further, the microneedles can be used for any chemical delivery, sensing, combinatorial chemistry, and microfluidic connectors.

Microneedle devices and manufacturing methods for the microneedles are described in the following patent applications, U.S. Ser. No. 09/095,221 filed on Jun. 10, 1998, U.S. Ser. No. 09/448,107 filed on Nov. 23, 1999, U.S. Ser. No. 09/452,979 filed on Dec. 2, 1999 and U.S. Ser. No. 09/453,109 filed on Dec. 2, 1999, all of which are incorporated herein by reference in their entirety.

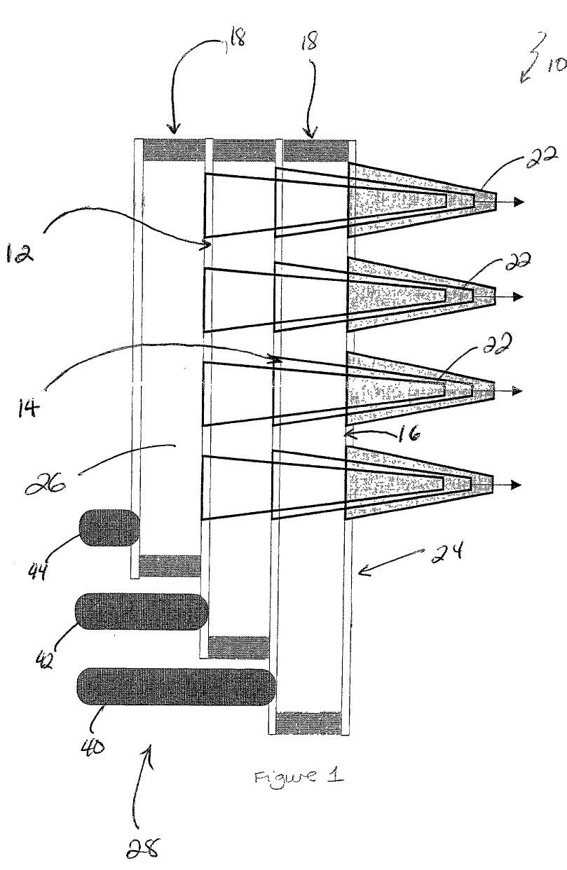

Referring to FIG. 1, a stacked system 10 of multiple injection of fluids is illustrated. The stacked system 10 includes a first array 16, a second array 14 and a third array 12. Each array 12, 14, 16 fits into the one below. While three arrays are shown, it is within the scope of the invention that any number of arrays can be used. Each array 12, 14, 16 can have a plurality of needles 22 mounted to a housing 24. The arrays 12, 14, 16 are sealed together using a sealing layer 18. For example, the seal 18 is created by a laminated adhesive layer between levels. Chambers are formed in the housing 24 or layered structure to form reservoirs 26. Each chamber is connected to a fluid or pressure source 28, such as a channel or a reservoir, to inject fluid into the system 10 or through a biological barrier. For example, arrays 12, 14 and 16 can be connected to fluid sources 40, 42, 44 respectively. Each fluid source 40, 42, 44 can contain fluids distinct from each other. Fluids can be injected sequentially, as in the use for injecting drugs, or simultaneously to obtain mixing of the fluids for dispensing applications. The reservoir 26 furthest away from the injection site, illustrated as array 12, can contain a buffer solution for washing out the needles and minimizing cross contamination.

When the needles of the first needle array 16 penetrate a biological barrier, different drugs (or multiple doses of the same drug), or different concentrations can be injected simultaneously or sequentially across a biological barrier, such as a tissue surface, for example.

When the first needle array 16 is located in proximity to a target substrate, the system can be used for dispensing such materials, for example, as oligonucleotides or cDNA for DNA arrays, peptides or proteins for proteomics or diagnostics, and adhesives for electronic assembly. The system 10 can also be used for multiple color ink-jet printing by placing different colors within each reservoir 28 and adding the appropriate actuators to manipulate the colors through the system 10.

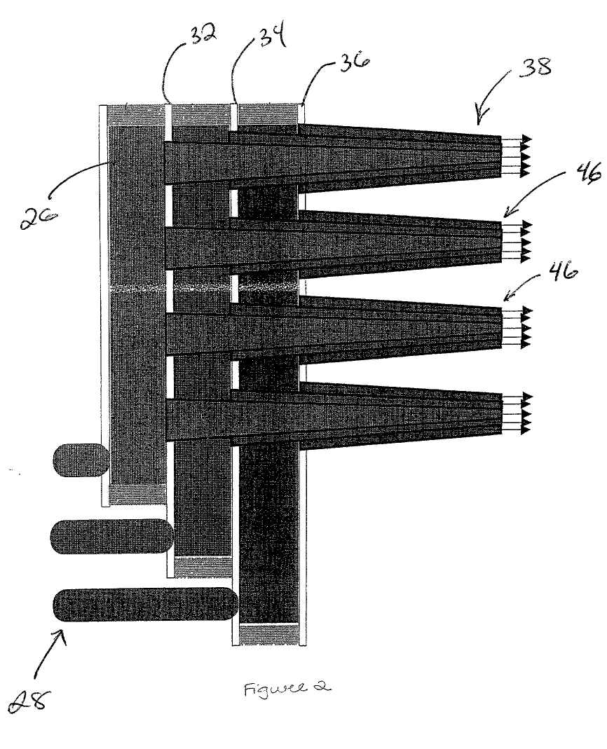

Referring to FIG. 2, a stacked system 30 with concentric needles 38 is illustrated. By providing needle arrays 32, 34, 36 of different lengths and diameters, a stacked system 30 with concentric needles 38 and concentric dispensing ends 46 located in approximately a single plane, can be assembled. Fluids from different reservoir 26 levels exit their respective needles 38 in the same plane. With these geometries, cross contamination is minimized, which is especially true for spotting and for chemical synthesis. Materials can be sequentially deposited onto an array, for example, to perform combinatorial synthesis. Under the appropriate conditions of pressure, flow rate, and substrate surface conditions, concentric rings of different materials can be deposited on the substrate. These rings can then be used to perform different chemical analyses on a target analyte. Also, the concentric rings can be formed in specific volume ratios for precise mixing of chemicals upon the addition of a buffer solution, or can be used in a diffusion controlled synthesis reaction. The concentric needles 38 can also be used for color printing where all colors are contiguous. In another embodiment, the needles 38 are arranged to produce spots in a line or square instead of rings. In this arrangement, the needles 38 can be rectangular to improve the rectangular geometry of the fluid output. Also, by using one or more needles 38 as a waveguide, photochemical reactions can be performed.

Further, the reservoirs 26 can be stacked vertically, or multiple reservoirs 26 can be attached to different portions of each needle array in the lateral direction.

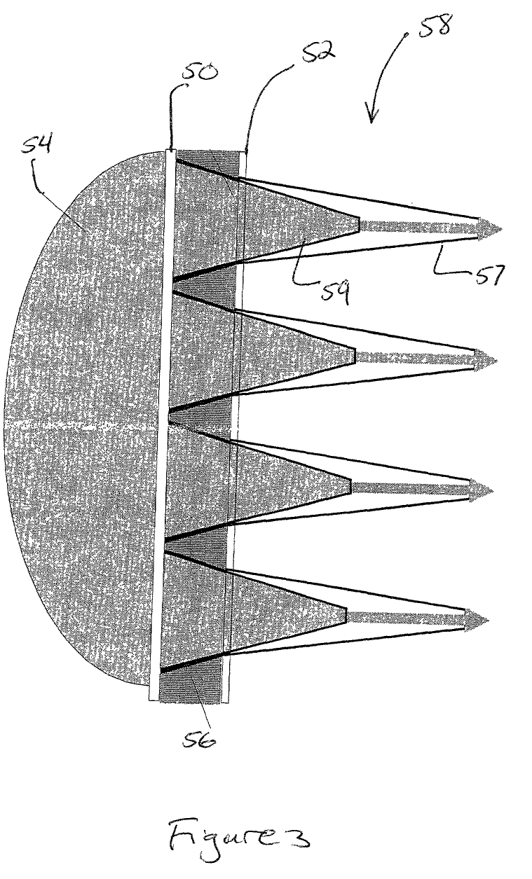

Referring to FIG. 3, a removably stacked dispensing needle system 58 is illustrated that includes a two-section system of needle arrays 50, 52. The first array 52 is a single needle array or piercing or contact needle array with a narrow taper. This array 52 is used to pierce the skin or tissue surface. It can be attached to a site as part of a patch arrangement such as a transdermal patch, or can be inserted at a site using an insertion tool, for example. The second array 50 of the system 58 includes a dispensing needle array. This array 50 can have needles of a slightly larger taper than the piercing needle array 52 to affect a seal against the piercing array 52. Alternatively, the array 50 can have a smaller taper angle which affects a seal down in the bore of the piercing array 52. This design allows for a metal-metal seal between the interior edge of the piercing needles 52 and the basal edge of the delivery needles 50. By designing the second array 50 with a taper geometry different than that of the first array 52, a seal between the arrays 52, 50, preferably a metal-metal seal, can be achieved. The first 52 and second 52 needle arrays can alternately have the same taper geometry.

The needles 59 of the second array 50 can be made from a different, preferably softer, material than the piercing needles 57 to affect a good seal. For example, the first needle array 52 can be made from NiFe, a relatively hard material, while the second array 50 can be made from gold (Au), a relatively soft material. The quality of the metal-metal seal can be enhanced if deformation of the needles occurs when they are pushed together. The deformation can yield an improved metal-to-metal seal between the two arrays 52, 50.

The dispensing needles 59 can also penetrate a thin septum 56 or seal layer to improve the fluid seal between the first 52 and second 50 arrays. The septum 56 aids in preventing fluid leakage between the piercing or connecting needle array 52 and the dispensing needle array 50. The seal 56 of the dispensing needle array 50 is in communication with a reservoir 54 that allows a fluid or drug to be injected into the piercing needle 57 and hence into the skin. In an alternate embodiment, the dispensing needles 59 can be a needle stack, as described previously.

The piercing needle arrays 52 have tip diameters of approximately 30-80 μm and have corresponding basal diameters of approximately 80-160 μm. Needles in the piercing needle array 52 can have lengths that range from between approximately 100-1000 μm with a preferred range between approximately 350-500 μm. For the delivery needle array 50, short tubes or highly tapered cones are used, typically in lengths of approximately 100-200 μm.

In a preferred embodiment, the piercing needle array 52 is inserted into an injection site. A dispensing needle array 50, in communication with a reservoir 54, is inserted within the piercing needle array 52. Fluid from the reservoir 54 can then be introduced through the dispersing array 50 and piercing array 52 and into the injection site. The dispensing needle array 50 can then be removed from the piercing needle array 52 and replaced with subsequent dispensing needle arrays. Such an arrangement of the dispensing needle system 58 avoids the necessity for the removal and reintroduction of a piercing needle array 52 to an injection site. In an alternate embodiment, the dispensing needle system 58 includes a plurality of stacked dispensing arrays 50 to allow for mixing or sequential injection of fluids.

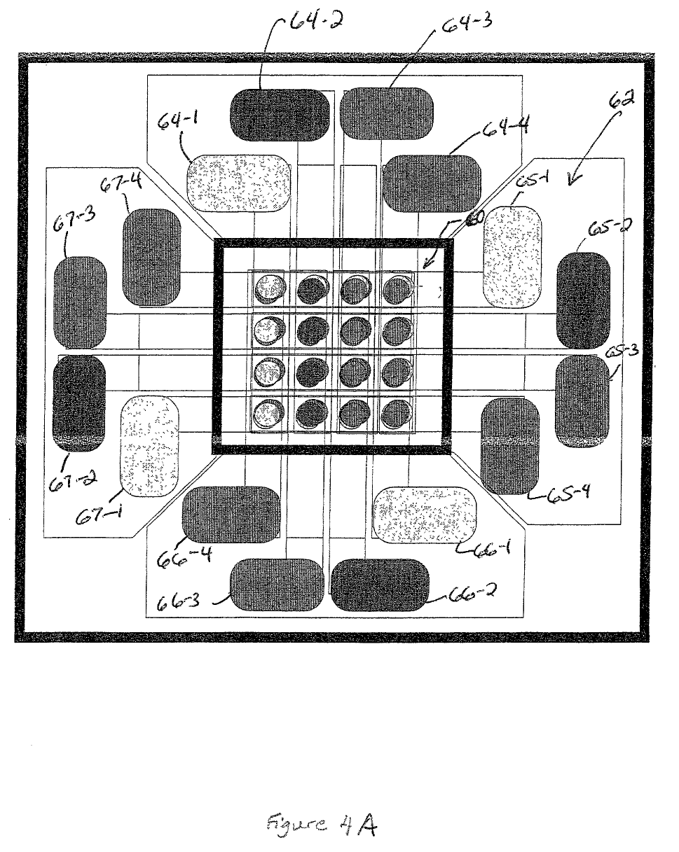

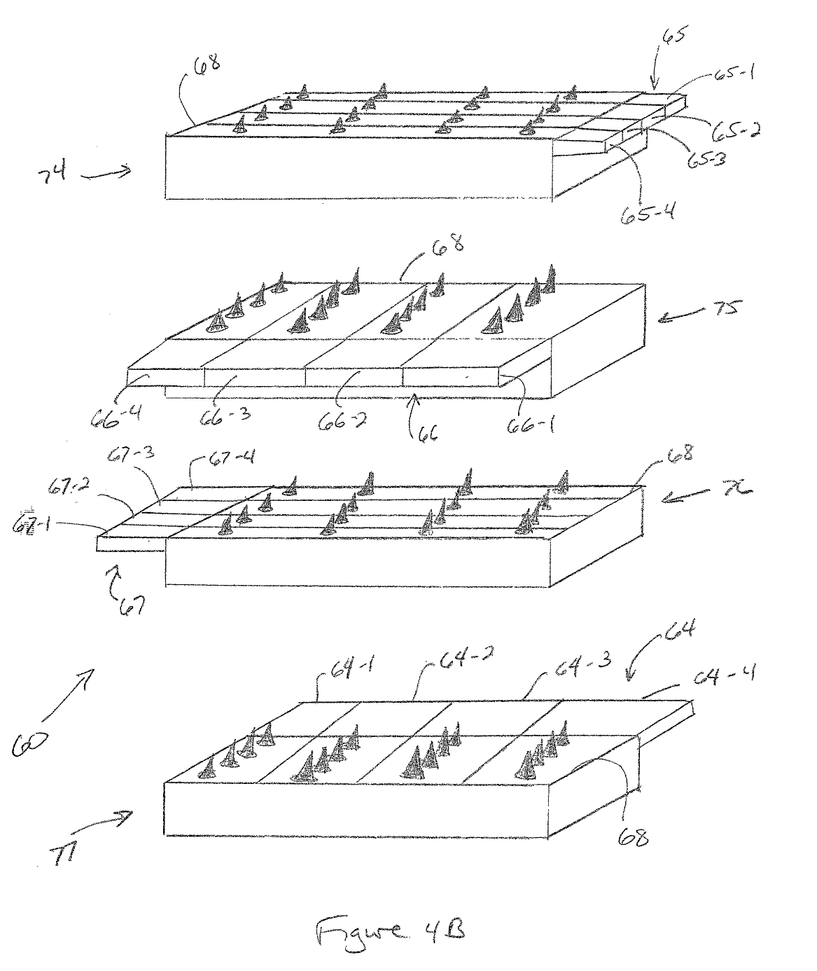

Referring to FIG. 4A, fluid distribution and combinatorial chemistry through a stacked microneedle array 60 can be accomplished. By stacking needle arrays coupled through channels fabricated in the laminated structures, different chemicals can be distributed, either sequentially or simultaneously, to different needles. These chemicals can be spotted onto a planar surface or injected into a volume. In a preferred embodiment the same four chemicals (for example, the four bases that make up DNA, or RNA) located in first chemical reservoirs 64-1 through 64-4, second chemical reservoirs 65-1 through 65-4, third chemical reservoirs 66-1 through 66-4 and fourth chemical reservoirs 67-1 through 67-4, are connected on four levels to the needle arrays. By arranging each needle array and reservoir in an orthogonal manner and locating the reservoirs 62 around the perimeter, a number of chemical combinations can be synthesized by sequentially activating different rows of needles on different levels. By changing, for example, the reservoir orientation, or by adding additional chemicals, more combinations can be achieved.

FIG. 4B illustrates an exploded, perspective view of the stacked microneedle array 60. The stacked microneedle array 60 has four separate arrays 74, 75, 76, 77. Each array 74, 75, 76, 77 includes a reservoir portion 64, 65, 66, 67, respectively. Each array 74, 75, 76, 77 is oriented within the microneedle array 60 such that the reservoir portion of each subsequently stacked array is rotated at an angle of 90° with respect to the array above the subsequently stacked array. For example, when stacking array 75 with array 74, the reservoir 66 of array 75 is oriented at a 90° angle relative to the reservoir 65 of array 74. This arrangement of arrays 74, 75, 76, 77 allows for the unique combination of chemicals or fluids located within the reservoirs. While four arrays 74, 75, 76, 77 are shown in FIG. 4B, a plurality of arrays can be used.

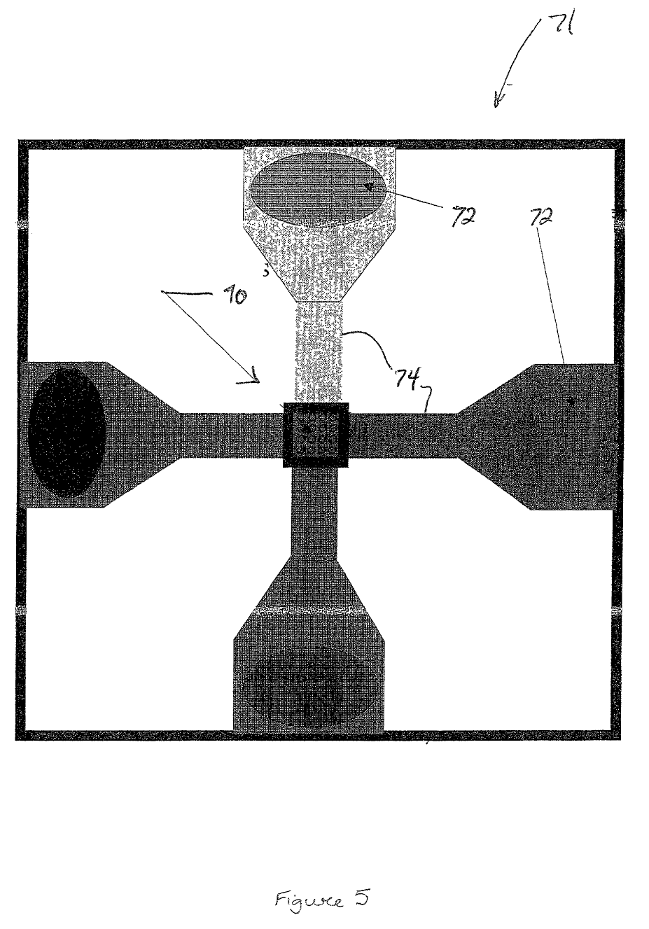

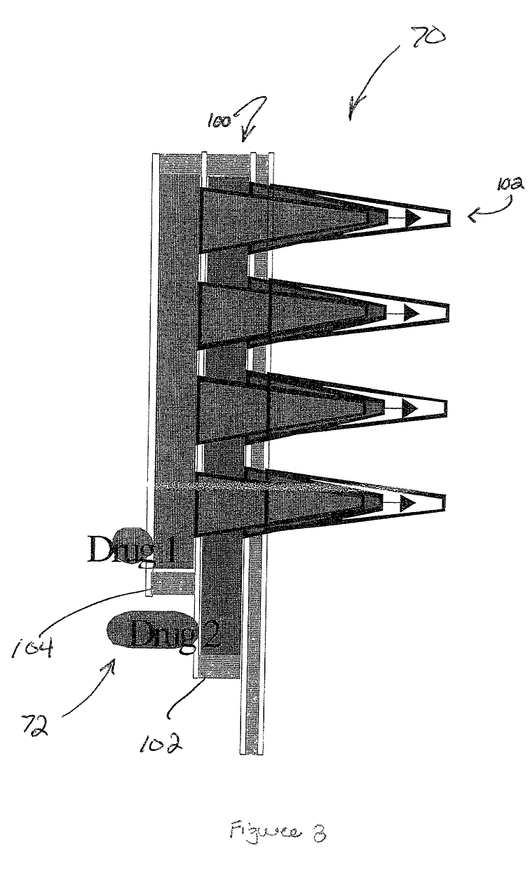

A delivery module 71, illustrated in FIGS. 5 and 8, allow multiple injections using a stacked needle array 70. By using channels 74 and membrane bubbles or reservoirs 72 on a needle array 70, a multiple dose drug injection module can be assembled. Each needle array layer consists of a microneedle array laminated to a polymer material such as, for example, Kapton or Mylar, with channels 74 cut into the polymer material to guide fluid to the needle array. The channels can be molded, embossed or die cut, for example. Each channel 74 of each array is then in communication with a membrane bubble or other reservoir 72 which holds a fluid or drug. By applying mechanical pressure, such as a user's thumb, or alternatively by connecting the reservoir to an external fluid source, a drug may be forced down the channel 74, through the needle array 70, and into the skin or other biological barrier. As illustrated in FIG. 5, the stacked needle array 70 includes four separate arrays, each array in fluid communication with an individual reservoir 72, in a preferred embodiment. Alternately, a plurality of needle arrays can be stacked together. Preferably, a maximum of eight separate arrays can be stacked together, for example.

As shown in FIG. 8, the stacked needle array 70 arrangement can be used in conjunction with separate piercing needles 102 which can act as a semi-permanent “catheter”. The piercing needles can be placed in a patient in conjunction with secondary needle arrays 100. Subsequent arrays can then be used without removal of the piercing array 102. Further, the stacked needle array 70 can include multiple drugs or multiple doses of the same drug delivered. Some applications include, for example, insulin injection, HIV drugs, and battlefield injections of pain-killers, and stimulants. In another embodiment, the system, for example, can be thumb activated. Other applications of the embodiment include its use in industrial applications, such as the injection of fluids into wings, plastics and foods, for example.

FIG. 8 illustrates a cross-sectional view of the stacked needle array 70 where the array 70 includes a piercing array 102 and a secondary needle array 100. In the secondary needle array 100, only two delivery arrays 102, 104 are shown. Preferably, the secondary needle array 100 includes four delivery arrays, each array connected to a separate reservoir 72.

The delivery module 71 has a number of drug reservoirs 72 fabricated in a laminated structure that also contains the delivery needles 100. This laminated structure utilizes a coaxial needle structure to minimize dead volume and cross- contamination. By using inexpensive laminated plastics, the delivery module 71 can be inexpensive to manufacture, disposable, and customizable for different delivery protocols.

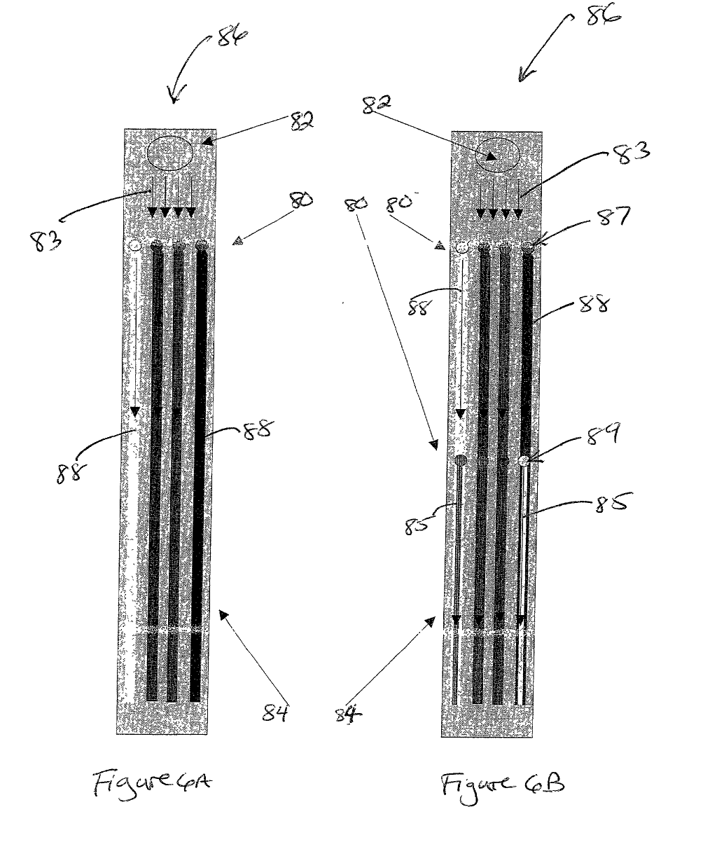

Referring to FIGS. 6A and 6B, laminar injection for diffusion controlled mixing can be accomplished using a needle array in conjunction with microfluid channels 84. It has been observed that in microfluidic systems of low Reynolds number, fluids can travel in streams of separate composition that mix only by diffusion. This phenomenon allows some unique chemical and fabrication processes to be implemented. Microneedles provide an ideal injection system for these laminar flow processes.

A microfluid system 86 includes a channel 84 and at least one microneedle array 80 for injecting streams 88 of fluid into the channel 84. By creating a microfluidic structure 86 with a mixing channel 84 and a microneedle array 80, fluid can be readily injected into a flowing buffer 83 in discrete streams 88 in the mixing channel 84. Buffer 82 is first injected into the channel 84 and acts to carry later injected material through the channel 84. Volumetric amounts and width of the streams 88 are controlled by, for example, the needle dimensions and pressure in the array 80. Stacked needle systems of the sort described herein can be used to inject different chemicals or to perform multiple injections of the same chemical for repeat experiments. Replaceable needle arrays can be mated to needles that are permanently installed in the microfluidic system 86. FIG. 6A illustrates the use of a single microneedle array 80 with a channel 84. A buffer 82 is first injected into the channel 84 to create a fluid flow within the channel 84. The microneedle array 80 then injects streams 88 into the channel 84, which can mix by diffusion. FIG. 6B illustrates the use of a plurality of needle arrays within the channel 84. A first needle array 87 is used to inject a first set of streams 88 into the channel 84. A second needle array 89, located distal to the first needle array 87, then injects a second set of streams 88 into the first set of streams 88, thereby creating a mixing between the first 88 and second 85 streams. Needle arrays located in tandem or in a stacked configuration can be used to sequentially inject different chemicals into the same stream of enhanced mixing control and sequential in-line chemical reactions.

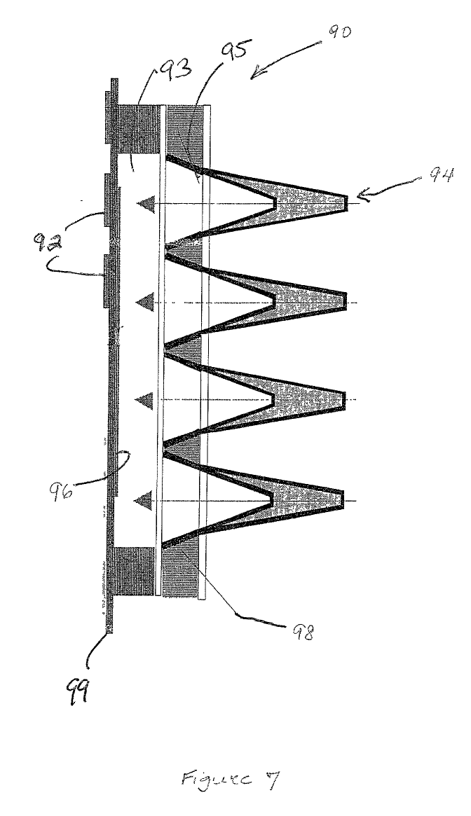

Referring to FIG. 7, a replaceable sensor module 90 in accordance with the present invention is illustrated. In combination with a sensing array 95 and a piercing needle array 94, a replaceable sensor module 90 can be fabricated. With the sensor module 90, fluid can be extracted from the dermal layers and analyzed. The sensing array 95, fits into the piercing array 94 and forms a seal 98. This sensing array 95 is mounted onto a chamber 93 to contain the extracted fluid. The top layer of this chamber is formed by a flexible circuit that contains a sensor chip 96, or in the alternative a plurality of sensor chips, that is electronically connected to a circuit board 99. The sensor chip 96 can be electrodes having a glucose oxyidase coating. When the piercing array 94 is introduced to a site, fluid such as interstitial fluid or blood from the site travels through the piercing array 94 and into the sensing array 95. When the fluid contacts the sensor chips 96, a voltage potential is created on the sensing array 95.

The chip 96 can be wire-bonded, flip-chip mounted, or tab bonded to the board 99, or formed directly onto the board 99 using passive electrodes or thin film electronics. Connections to the chip 96 are brought outside the chamber 93 or brought through the board 99 using vias. Additional control chips 92 and connections can be mounted on the board to form a functional module 90.

Interstitial fluid (ISF) can be sampled through the piercing needle array 94 by applying suction to the needles 94 to pull fluid though piercing needle array 94 to the sensing array 95. Alternatively, pressure can be applied externally to force fluid up piercing needles 94. The extracted fluid is sensed utilizing the sensing array 95 as the sensing element. By filling or coating the sensing array 95 with the appropriate enzymes for glucose sensing, the enzymes can readily interact with ISF brought up through the piercing needle array 94. The sensing array 95 and piercing needles 94 form opposite electrodes for measuring the electrochemical activity between the enzymes and ISF. The small needle area allow the sensitive measurement of target species in the interstitial fluid. Read-out and control chips 92 can be incorporated into the sensing module 90 for readout. The sensors 96 can detect levels of various injectable drugs being used by a subject to enhance performance. In addition, blood glucose and blood chemistry can be monitored using the sensor system 90.

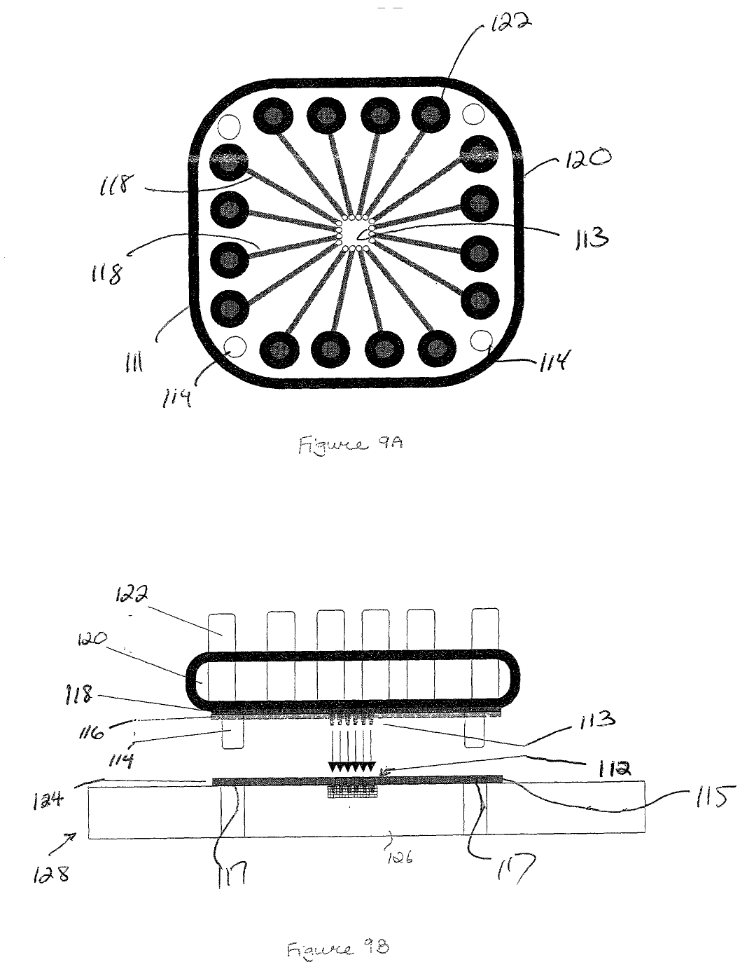

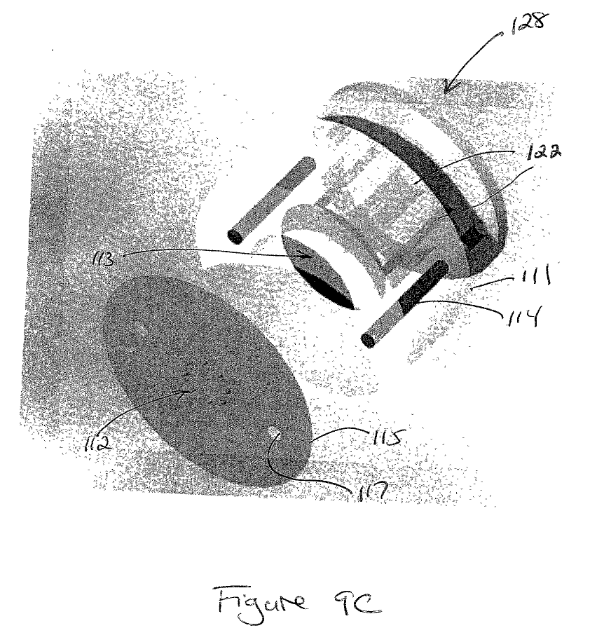

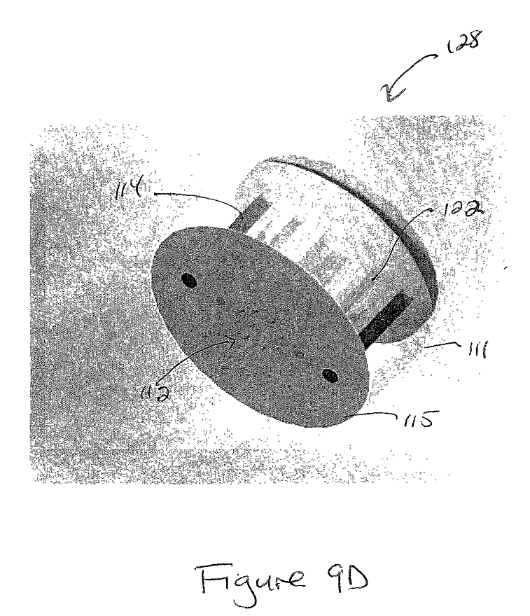

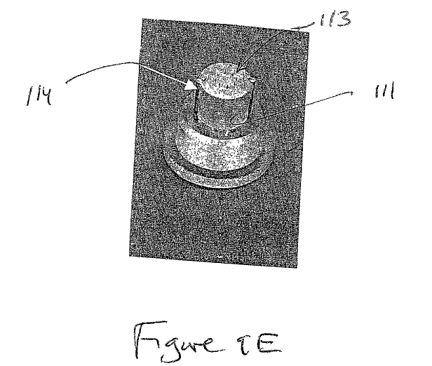

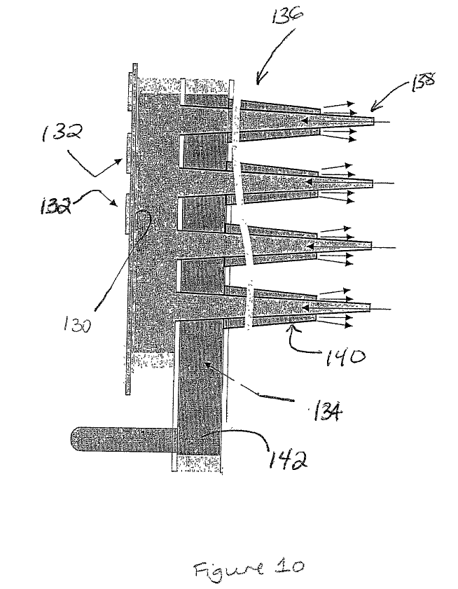

Referring to FIGS. 9A and 9B, a preferred embodiment of a microfluidic connector 120 is illustrated. An important problem in microfluidics is the reliable, low dead volume connection to microfluidic systems where the volumes may be in the order of microliters or nanoliters and the fluid elements are of the order of tens of microns. By using a stacked needle array, multiple connections can be made to a microfluidic system.

An array of microneedles 112 is laminated into the microfluidic system 128 in such a manner as each needle in the array 112 corresponds to an individual needle in a needle array 113 in the connector 110. The array 112 is bonded in such a manner as to form the top layer of the system 128 using a laminated seal 124. On the connector portion 120, a secondary needle array 113, fabricated with a wider taper and perhaps, from a more compliant material, is used to connect to the needles 112 on the microfluidic system 128. A compliant material is used to form the overall seal 116 around the array 113. Each needle in the secondary array 113 is connected to a die cut or molded channel 118 which fans out to a macroscopic tubing connector 122 mounted in the connector body 120. Locating pins 114 in conjunction alignment holes 117 with are used to coarsely align the connection between the microfluid system 128 and the connector portion 120. The secondary needle arrays 112 can be stacked to achieve mixing or a higher density.

FIGS. 9C and 9D illustrate perspective views of a microfluidic system128. The body of the device 150 is less than one cm in diameter and can be made from polycarbonate, for example. The inner 111 and outer 115 sections hold the two needle arrays 112, 113. Guiding pins 114 are included to prealign the arrays 112, 113 to allow for proper insertion. The guide pins are mated with precision alignment holes 117 on the needle array 112. The arrays 112, 113 are held to the connector 120 with adhesives. Fluids are delivered through a port on the upper surface of the insert. The two sections of the device can be machined to provide a precision, coaxial fit with a keyway. The needle arrays 112, 113 are fabricated with different outer diameters to fit the inside and outside diameters of the coaxial needle modules 111, 115.

To use the microfluidic connector, the outer section 115 is attached to an adhesive or velcro strap. The needles 112 are then pushed into skin or tissue 126 using manual force, and the strap tightened to hold the device 115 in place. The inner cylinder 111 include one or more sealed reservoirs of fluids or drugs that can be manually actuated, forcing fluid down through the set of coaxial needles 113, through the insertion needles 112, and into the skin or tissue. The seal between the piercing needle array 112 and the delivery array 113 can be created by a metal-to metal seal between the needles and can be assisted by an additional polymeric sealing layer 116. The device can include a locking mechanism that retains pressure between the two needle arrays 113, 115 and maintains a liquid seal between them.

In an alternate embodiment, illustrated in FIG. 9E, the location of the alignment pins 114 on the delivery needle component 111 are located on the perimeter of the component 111. Such positioning results in an increase of the surface area available to a sealing adhesive thereby preventing leakage between the needle arrays 113, 112.

Referring to FIG. 10, sensing and delivery functions are combined onto a single coaxial configuration. FIG. 10 illustrates a sensor module 136 having a sensing needle array 138 and a delivery needle array 140. The sensing needle array 138 includes control chips 132 and sensor chip 130. The delivery needle array 140 includes a chamber 142 to hold a fluid 134, such as a drug, for example. The sensing needle array 138 of the module 136 is engaged with an injection site. Fluid from the injection site such as interstitial fluid, enters the piercing needle array 138 and contacts the sensor chip 130. The sensor chip 130 can be coated with glucose oxidase, such that contact with the fluid creates a voltage potential. This potential can be used to drive or pump fluid through the delivery needle array 140 and into the injection site.

A combination of the delivery module and sensor module, with additional coaxial needles in a stack, form a fully integrated sensing and delivery system, shown in FIG. 10. In this system, needles of different lengths are used to separate the sensing and delivery functions. Support modules can include pumps, valves, and electronics to monitor blood chemistry and control drug delivery.

While this invention has been particularly shown and described with references to preferred embodiments thereof, it will be understood by those skilled in the art that various changes in form and details may be made therein without departing from the scope of the invention encompassed by the appended claims.

What is claimed is:

1. A device for fluid delivery comprising:
a plurality of needles having channels defining a fluid flow path; and
at least one chamber which is selectably in fluid communication with the plurality of needles.

2. The device of claim 1 wherein the plurality of needles are connected and aligned along a common axis.

3. The device of claim 1 wherein the plurality of needles are detachably connected.

4. The device of claim 1 wherein a plurality of fluids can be delivered simultaneously.

5. The device of claim 1 wherein a plurality of fluids can be delivered sequentially.

6. The device of claim 1 wherein the plurality of needles comprise a plurality of dispensing ends located in approximately the same plane.

7. The device of claim 1 wherein the plurality of needles comprises a contact needle array.

8. The device of claim 7 further comprising at least one dispensing needle array that is mounted within the contact needle array.

9. The device of claim 8 wherein the at least one dispensing needle array comprises at least one reservoir.

10. The device of claim 1 further comprising a sensing array.

11. The device of claim 10 wherein the sensing array comprises a chamber and at least one sensor circuit.

12. The device of claim 1 wherein the contact array comprises at least one reservoir in fluid communication with the contact array.

13. A method for fluid delivery comprising:
providing a plurality of needles having channels, defining a fluid flow path; and
connecting the plurality of needles.

14. The method of claim 13 further comprising connecting the plurality of needles such that the channels are aligned along a common axis.

15. The method of claim 14 further comprising detachably connecting the plurality of needles.

16. The method of claim 13 further comprising connecting the plurality of needles to a fluid source.

17. The method of claim 13 further comprising connecting the needles to a plurality of fluid sources.

18. The method of claim 13 further comprising mixing a plurality of fluids.

19. The method of claim 13 further comprising delivering a fluid across a biological barrier.

20. The method of claim 19 further comprising delivering a drug to a patient across tissue of the patient.

21. The method of claim 13 further comprising providing a first needle array connected to a first reservoir and a first fluid source and a second needle array connected to a second reservoir and a second fluid source.

22. The method of claim 13 further comprising providing a pump that pumps fluid through the needles.

23. The method of claim 13 further comprising providing a sensor that detects a fluid.

24. The method of claim 13 further comprising controlling fluid flow with a control circuit.

25. The method of claim 13 further comprising reacting a first fluid and a second fluid.

26. The method of claim 13 further comprising delivering a plurality of drugs through the skin of a patient.

27. The method of claim 13 further comprising removing fluid from a site with the needle array.

28. A drug flow device comprising:
a first needle array; and
a second needle array in fluid communication with the first needle array such that a drug can be conducted along a path through the first needle array and the second needle array.

29. The device of claim 28 further comprising alignment pins, wherein the alignment pins provide alignment between the first needle array and the secondary needle array.

30. The device of claim 28 further comprising needles having distal ends with a diameter in a range of 30-80 microns.

31. The device of claim 28 wherein the needles having a length in a range of 100-1000 microns.

32. The device of claim 28 wherein the first array has a first taper and the second array has a second taper.

33. The device of claim 28 further comprising a sensor circuit that measures a fluid in the device.

34. The device of claim 28 further comprising an actuator that controls fluid flow in the device.

35. The device of claim 28 further comprising a control circuit that controls the device.

36. The device of claim 28 further comprising a plurality of fluid pathways extending through a plurality of coaxially positioned needles.

37. The device of claim 28 further comprising a pump to deliver fluid through the device.

38. The device of claim 28 further comprising a seal between the first array and the second array.

39. The device of claim 28 further comprising a circuit board mounted on the device.

40. The device of claim 28 further comprising a laminated structure having at least three arrays of metal needles.
